(19) United States
(12) Patent Application Publication
(10) Pub. No.: US 2021/0100887 A1
(43) Pub. Date: Apr. 8, 2021

(54) RECOMBINANT FUSION PROTEIN AND IMMUNOGENIC COMPOSITION

(71) Applicant: Reber Genetics Co., Ltd., Taipei City (TW)

(72) Inventors: Wei-Jen Chen, Changhua County (TW); Chia-Jung Chang, Kaohsiung City (TW); Pei-Yin Wu, Yi-Lan County (TW)

(73) Assignee: Reber Genetics Co., Ltd., Taipei City (TW)

(21) Appl. No.: 17/037,732

(22) Filed: Sep. 30, 2020

Related U.S. Application Data
(60) Provisional application No. 62/910,474, filed on Oct. 4, 2019.

(51) Int. Cl. A61K 39/00; C07K 14/435; C07K 14/705; C07K 14/21; C12N 9/64; A61P 35/00

(52) U.S. Cl. CPC A61K 39/001158; C07K 14/435; C07K 14/70521; C07K 14/21; C12N 9/6472; A61K 2039/575; A61P 35/00; C07K 2319/40; C07K 2319/04; A61K 2039/55561; A61K 2039/55577; C12Y 304/22034

(57) ABSTRACT

A recombinant fusion protein includes a receptor associated protein 1 (RAP1), a cluster of differentiation 28 (CD28)-Pseudomonas exotoxin A translocation domain (PEt) fusion polypeptide, a legumain protein and a target peptide. The RAP1 is located at the N-terminus of the recombinant fusion protein. The CD28-PEt fusion polypeptide is located at the C-terminus of the RAP1. The legumain protein is located at the C-terminus of the CD28-PEt fusion polypeptide. The target peptide is located at the C-terminus of the legumain protein. In another embodiment of the present disclosure, an immunogenic composition is provided. The immunogenic composition including the recombinant fusion protein and an adjuvant is used for inducing specific immune responses in a subject with cancer, whereby the risk of cancer metastasis and recurrence for the subject may be successfully reduced.

### CROSS-REFERENCE TO RELATED APPLICATION

This application claims the priority benefit of U.S. provisional application Ser. No. 62/910,474, filed on Oct. 4, 2019. The entirety of the above-mentioned patent application is hereby incorporated by reference herein and made a part of this specification.

### BACKGROUND

### Technical Field

The present invention generally relates to a recombinant fusion protein an immunogenic composition, in particular, relates to an immunogenic composition including the recombinant fusion protein that can effectively induce specific immune responses in a subject with cancer.

### Description of Related Art

Legumain is overexpressed in a variety of tumors and is more significant at later stages of the tumor and at the time of metastasis. Therefore, legumain overexpression is considered to be associated with the risk of postoperative metastasis and recurrence. For example, Legumain is overexpressed in dog with breast tumor, and the main treatment is surgical resection. However, in clinical it has been found that the rate of metastasis and recurrence after surgical removal of breast tumor is quite high.

### SUMMARY

Accordingly, the present disclosure is directed to an immunogenic composition including a recombinant fusion protein that may be used to effectively induce specific immune responses in a subject with cancer, and may reduce the risk of cancer metastasis and recurrence for the subject.

In accordance with some embodiments of the present disclosure, a recombinant fusion protein is provided. The recombinant fusion protein includes a receptor associated protein 1 (RAP1), a cluster of differentiation 28 (CD28)-Pseudomonas exotoxin A translocation domain (PEt) fusion polypeptide, a legumain protein and a target peptide. The RAP1 is located at the N-terminus of the recombinant fusion protein. The CD28-PEt fusion polypeptide is located at the C-terminus of the RAP1. The legumain protein is located at the C-terminus of the CD28-PEt fusion polypeptide. The target peptide is located at the C-terminus of the legumain protein.

In the above embodiment, the recombinant fusion protein includes an amino acid sequence of SEQ ID NO:1.

In the above embodiment, the recombinant fusion protein includes an amino acid sequence encoded by a nucleotide sequence of SEQ ID NO:2.

In the above embodiment, the legumain protein is a human recombinant legumain protein.

In the above embodiment, the target peptide is an endoplasmic reticulum (ER) retention sequence.

In another embodiment of the present disclosure, an immunogenic composition is provided. The immunogenic composition is used for inducing specific immune responses in a subject with cancer. The immunogenic composition includes the recombinant fusion protein and an adjuvant.

In the above embodiment, the adjuvant includes CpG oligodeoxynucleotides (CpG ODN) or saponin-based adjuvant.

In the above embodiment, a concentration of the CpG ODN is about 0.2 mg/ml.

In the above embodiment, a concentration of the saponin-based adjuvant is about 0.2 mg/ml.

In the above embodiment, a ratio of the recombinant fusion protein to the adjuvant is 2.5:1 by weight.

In the above embodiment, the subject includes human and non-human animal.

In the above embodiment, the cancer includes legumain-overexpressed cancer.

In the above embodiment, the legumain-overexpressed cancer includes breast cancer, mammary tumor, prostate cancer, stomach cancer, colorectal cancer, ovarian cancer, and melanoma.

Based on the above, the present invention provides a recombinant fusion protein and an immunogenic composition including the recombinant fusion protein. The recombinant fusion protein includes RAP1 locating at the N-terminus of the recombinant fusion protein, a CD28-PEt fusion polypeptide locating at the C-terminus of the RAP1, a legumain protein locating at the C-terminus of the CD28-PEt fusion polypeptide and a target peptide locating at the C-terminus of the legumain protein. By adding the recombinant fusion protein of the present disclosure in the immunogenic composition for vaccination, effectively specific immune responses may be successfully induced in a subject with cancer, hence reducing the risk of cancer metastasis and recurrence for the subject.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A is illustrating the results from ELISA for detecting the presence of legumain antibody in mice for different test groups from Example 2.

FIG. 1B to FIG. 1D illustrate the results from IVIS for detecting the 4T1/luc mouse mammary tumor cells in mice for different test groups from Example 2.

FIG. 2A is illustrating the results from ELISA for detecting the presence of legumain antibody in dogs for different test groups from Example 3.

FIG. 2B is illustrating the results for detecting the volume of mammary tumor in dogs from Example 3.

### DESCRIPTION OF THE EMBODIMENTS

Currently, Legumain is found to be overexpressed in a variety of tumors and is more significant at later stages of the tumor and at the time of metastasis. In addition, legumain overexpression is considered to be associated with the risk of postoperative metastasis and recurrence. For example, Legumain is overexpressed in dogs with mammary tumors, and in clinical it has been found that the rate of metastasis and recurrence after surgical removal of the mammary tumor is very high.

The present disclosure is directed to a recombinant fusion protein including an amino acid sequence of SEQ ID NO:1 and an immunogenic composition including the recombinant fusion protein and an adjuvant for effectively induce specific immune responses in a subject with cancer. In some exemplary embodiments, the recombinant fusion protein may at least include a receptor associated protein 1 (RAP1), a cluster of differentiation 28 (CD28)-Pseudomonas exotoxin A translocation domain (PEt) fusion polypeptide, a legumain protein and a target peptide. The RAP1 may be located at the N-terminus of the recombinant fusion protein. The CD28-PEt fusion polypeptide may be located at the C-terminus of the RAP1. The legumain protein may be located at the C-terminus of the CD28-PEt fusion polypeptide. The target peptide may be located at the C-terminus of the legumain protein.

In one exemplary embodiment, the recombinant fusion protein may include an amino acid sequence encoded by a nucleotide sequence of SEQ ID NO:2. However, the disclosure is not limited thereto.

The RAP1 having a molecular weight of 39 kDa is an ER resident protein. The RAP1 is also an antagonist and molecular chaperone that binds tightly to low-density lipoprotein receptor family members, for example, low density lipoprotein receptor-related protein 1 (LRP1), also known as cluster of differentiation 91 (CD91).

The Pseudomonas exotoxin A (PE) protein is the most toxic virulence factor of this bacterium. The PE protein can be divided into Ia domain (amino acid sequence 1-252), II domain (amino acid 253-364), Ib domain (amino acid sequence 365-404) and III domain (amino acid sequence 405-613). In some embodiments, the amino acid sequence 268-313 of the PE protein having PE translocation domain (PEt) may be used as part of the recombinant fusion protein. However, the disclosure is not limited thereto. In other embodiments, the amino acid sequence the PE protein having PEt may also be used as part of the recombinant fusion protein.

The CD28-PEt fusion polypeptide may contain CD28 conserved region and PE translocation domain (PEt), and the PEt may be located at the C-terminus of the CD28 conserved region. The CD28 conserved region is an epitope for inducing CD28 agonist antibody. The CD28-PEt fusion polypeptide used as immunostimulator may raise much higher IgG titer specific to CD28 (CD28 agonist antibody) and then sensitize both CD4+ and CD8+ T cells.

The legumain protein is a cysteine endopeptidase belonging to the peptidase family C13. The legumain protein is a very important clinical indicator in tumors and is overexpressed in a variety of tumors, including breast cancer, mammary tumor, prostate cancer, stomach cancer, colorectal cancer, ovarian cancer and melanoma in humans. The legumain protein is also overexpressed in anal sac carcinoma, lymphoma, mammary gland tumor, mast cell tumor, oral malignant melanoma, osteosarcoma, soft tissue sarcoma (fibrosarcoma, peripheral nerve sheath tumor and others) and splenic hemangiosarcoma in non-humans (such as dog, but is not limited thereto). In addition, because the expression level of the legumain protein in stage III-IV cancer is higher than that in stage I-II cancer, overexpression of the legumain protein may be related to clinical stage of cancer. Specifically, overexpressed legumain may seriously affect the process of antigen presentation, thereby affecting the activation of MHC II, but is not limited thereto. Overexpressed legumain may also affect the activation of other proteases on the cell surface. For example, tumor-associated macrophages (TAMs) such as M2 TAMs can establish a microenvironment suitable for tumor growth by overexpressing the legumain protein and through the above mechanism.

In one exemplary embodiment, the legumain protein may be a human (Homo sapiens) recombinant legumain protein. However, the disclosure is not limited thereto. In other exemplary embodiment, other species of legumain protein having other amino acid sequence(s) may be used.

In one exemplary embodiment, the target peptide may contain an endoplasmic reticulum (ER) retention sequence, which assists translocation of an antigen from an endocytic compartment into ER and retains it in the lumen. The ER retention sequence may include the amino acid sequence of KDEL or RDEL. For example, the ER retention sequence may include the amino acid sequence of KDELKDELKDEL. However, the disclosure is not limited thereto.

In one exemplary embodiment, the subject may include human and non-human animal. For example, the subject may be a mouse, a cat or a dog. However, the disclosure is not limited thereto.

In one exemplary embodiment, the cancer may include legumain-overexpressed cancer. In other exemplary embodiment, the legumain-overexpressed cancer may include breast cancer, mammary tumor, prostate cancer, stomach cancer, colorectal cancer, ovarian cancer, and melanoma in humans. However, the disclosure is not limited thereto. In other exemplary embodiment, the legumain-overexpressed cancer may include anal sac carcinoma, lymphoma, mammary gland tumor, mast cell tumor, oral malignant melanoma, osteosarcoma, soft tissue sarcoma (fibrosarcoma, peripheral nerve sheath tumor and others) and splenic hemangiosarcoma in non-humans (such as dog, but is not limited thereto).

In one exemplary embodiment, the adjuvant may include CpG oligodeoxynucleotides (CpG ODN) or saponin-based adjuvant. For example, the saponin-based adjuvant may be a saponin-based veterinary vaccine adjuvant (VET-SAP). However, the disclosure is not limited thereto.

In one exemplary embodiment, in the immunogenic composition, a concentration of the CpG ODN is about 0.2 mg/ml. In some embodiments, in the immunogenic composition, a concentration of the saponin-based adjuvant is about 0.2 mg/ml.

In one exemplary embodiment, a ratio of the recombinant fusion protein to the adjuvant is 2.5:1 by weight. In some embodiments, a ratio of the recombinant fusion protein to the adjuvant is 12.5:1 by weight. In certain embodiments, a ratio of the recombinant fusion protein to the adjuvant is 1:1 by weight. By adjusting the ratio of the recombinant fusion protein to the adjuvant in such a range, effectively specific immune responses may be induced.

By designing the recombinant fusion protein to include at least a receptor associated protein 1 (RAP1), a cluster of differentiation 28 (CD28)-Pseudomonas exotoxin A translocation domain (PEt) fusion polypeptide, a legumain protein and a target peptide, effectively specific immune responses may be successfully induced in a subject with cancer by an immunogenic composition including the recombinant fusion protein described above, hence reducing the risk of cancer metastasis and recurrence for the subject.

### EXAMPLES

The following experimental examples are performed to prove that the immunogenic composition including the recombinant fusion protein of the present disclosure can successfully induce specific immune responses in a subject with cancer, so that the risk of cancer metastasis and recurrence for the subject can be reduced.

### Example 1: Preparation of the Recombinant Fusion Protein and the Immunogenic Composition Including the Recombinant Fusion Protein

[Expression of the Recombinant Fusion Protein]

In this example, 500 mL culture medium (125 μL kanamycin (100 mg/mL), 50 mL 20% glucose solution, 450 mL TSB medium) was prepared in a 2 L flask in a sterile hood. Next, the bacterial stock was inoculated to the 2 L flask containing 500 mL culture medium, and incubated with shaking (150 rpm) in a 30° C. incubator for 14˜18 hours. It should be noted that the above bacteria can express the recombinant fusion protein including the amino acid sequence of SEQ ID NO: 1. In other example, the bacteria above have a nucleotide sequence of SEQ ID NO: 2 which can encode the amino acid sequence of the recombinant fusion protein.

Preparation of a culture solution in the fermenter: after adding 48 g tryptone, 96 g yeast extract, 8.8 g KH₂PO₄, 37.6 g K₂HPO₄, 1 g NaCl, and 1 mL defoamer into the fermenter, adding water to 4 L and sterilizing at 121° C. for 20 min. After sterilization and lowering the temperature to 37° C., 100 mL carbon source solution (containing 16 g glucose and 64 g glycerol) and 1 mL kanamycin (100 mg/mL) were added into the fermenter.

The bacterial culture medium cultured overnight in the 2 L flask was added into the fermenter, and cultured under the following culture conditions: temperature is 37° C., rotation speed is 400 to 1000 rpm, pH is 7.0, value of dissolved oxygen (DO) is 20%, and ventilation is 3 ccm. The initial optical density at 600 nm wavelength (OD600) of the culture solution in the fermenter is between 0.01˜0.04.

After incubation for 6 hours, 4 mL of IPTG (1 M) was added to the fermenter for induction. After IPTG induction for more than 8 hours and the pH is greater than 7.5, a fermentation product can be collected.

[Collection of the Recombinant Fusion Protein]

The fermentation product was centrifuged at 8,000 rpm and 4° C. for 10 min. The supernatant was removed and the pellet was homogeneously resuspended by TNE buffer (50 mM Tris, 50 mM NaCl, 1 mM EDTA, pH 8.0)

After the cold water system was installed in a high-pressure cell crusher, the suspension was poured into the high-pressure cell crusher. The bacterial cells in the suspension were ruptured by the high-pressure cell crusher in the condition of 4° C. and 1,300 bar to obtain a cell lysate. Repeat this step 1 more time.

The cell lysate was centrifuged at 12,000 rpm and 4° C. for 30 min to collect a pellet.

The pellet was washed once with TNE-T buffer (50 mM Tris, 50 mM NaCl, 1 mM EDTA, 5% Triton X-100, pH 8.0) and centrifuged at 7,000 rpm for 30 min, and then removing the supernatant.

The pellet was washed twice with TNE-T buffer and centrifuged at 7,000 rpm for 20 min, and then removing the supernatant.

The pellet was washed 3 times with 2 M Urea-T buffer (2 M Urea, 50 mM Tris, 50 mM NaCl, 1 mM EDTA, 5% Triton X-100, pH 8.0) and centrifuged at 7,000 rpm for 15 min, then removing the supernatant.

The precipitate was washed with TNE buffer and centrifuged at 7,000 rpm for 30 minutes, and then the supernatant was removed to obtain an inclusion body. The inclusion body containing the recombinant fusion protein was stored in the −20° C.

[Purification of the Recombinant Fusion Protein]

Three grams (3 g) of inclusion body was homogeneously resuspended by 150 mL of binding buffer (8 M Urea, 10 mM potassium phosphate (K. Phosphate), pH 6.0)

After adding 3 mL of 0.5 M DTT, the resuspended inclusion body was poured into the high-pressure cell crusher, followed by high-pressure homogenization at a pressure of 1,300 bar. It should be noted that the resuspended inclusion body must be homogenized completely before pouring into the high-pressure cell crusher. After high-pressure homogenization, a lysate solution was placed on a shaker at 150 rpm and 37° C. for 16-24 hours.

Afterwards, the lysate solution was centrifuged at 7,000 rpm and 4° C. for 10 min.

The supernatant was collected and filtered by a 0.45 μm/0.22 μm filter, and the filtered semi-products containing the recombinant fusion protein were collected.

After balancing the 10-mL Q Sepharose column with a binding buffer (8 M Urea, 10 mM Potassium (K.) Phosphate, pH 6.0) of 5 times the column volumes (flow rate at 5 mL/min), 50 mL of the filtered semi-products was added to the Q Sepharose column.

Adding a 15% Elution buffer of 3 times the column volume to wash the column at a flow rate of 5 mL/min, and then collecting the washed samples. The Elution buffer contains 8M Urea, 10 mM K. Phosphate and 500 mM NaCl at pH 6.0.

Adding 3 times the column volume of 20% Elution buffer to wash the column at a flow rate of 5 mL/min, and then collecting the washed samples.

Adding 3 times the column volume of 100% Elution buffer to wash the column at a flow rate of 5 mL/min, and then collecting the purified semi-products containing the recombinant fusion protein.

[Refolding of the Recombinant Fusion Protein]

After adding 10 mg purified semi-product to a dialysis bag (30 kD cut-off), add 8 M Urea buffer (8 M Urea, 10 mM K. Phosphate, pH 6.0) to the dialysis bag to 10 mL.

The dialysis bag was placed in a container containing 5 L of 6 M Urea buffer (6 M Urea, 10 mM K. Phosphate, pH 6.0) for dialysis for 16 hours.

The dialysis bag was taken out and placed in a container containing 5 L of 4 M Urea buffer (4 M Urea, 10 mM K. Phosphate, pH 6.0) for dialysis for 4 hours.

The dialysis bag was taken out and placed in a container containing 5 L of 2 M Urea buffer (2 M Urea, 10 mM K. Phosphate, pH 6.0) for dialysis for 4 hours.

The dialysis bag was taken out and placed in a container containing 5 L of 1 M Urea buffer (1 M Urea, 10 mM K. Phosphate, pH 6.0) for dialysis for 16 hours.

The dialysis bag was taken out and placed in a container containing 5 L of 1×PBS (pH 7.4) for dialysis for 4 hours.

The dialysis bag was taken out and placed in a container containing 5 L of 1×PBS (pH 7.4) for dialysis for 4 hours.

After the refolded semi-product was taken out and filtered with a 0.22-μm filter, the recombinant fusion protein was obtained.

[Preparation of the Immunogenic Composition]

After 10 μL Neomycin and 100 μL CpG/VET-SAP (20 mg/mL) were added to 10 mL of the recombinant fusion protein to form a mixture, the mixture was sterile-filtered into a mixing tank with a 0.2-μm filter and stirred at 150 rpm for 5 minutes to obtain the immunogenic composition. In this experimental example, the recombinant fusion protein was uniformly mixed with CpG/VET-SAP, for example, in a ratio of 2.5:1. However, the disclosure is not limited thereto. In other experimental examples, recombinant fusion protein and CpG/VET-SAP can also be mixed uniformly in a ratio of 1:1 or 12.5:1. In some experimental example, the concentration of the CpG ODN is about 0.2 mg/ml. In other experimental example, the concentration of the saponin-based adjuvant is about 0.2 mg/ml.

### Example 2: Effect of the Immunogenic Composition on Animal Model of Mammary Tumor

In this experimental example, female BALB/c mice (7 weeks of age) were used as test animals and were injected with placebo or immunogenic composition by subcutaneous injection. Balb/c mice were divided into 3 groups and there are 3-10 mice in each group as shown in Table 1. Firstly, Groups A to C were injected with mouse mammary tumor cells (for example 1.4*10⁴ 4 T1-luc cells) by tail vein injection. 3, 10 and 17 days after injection of mouse mammary tumor cells, Group A was injected with buffer (as placebo), Group B was injected with the immunogenic composition including the recombinant fusion protein and CpG, Group C was injected with the immunogenic composition including the recombinant fusion protein and VET-SAP. It should be noted that 4T1 mouse mammary tumor cells is highly tumorigenic and invasive and can spontaneously metastasize from the primary tumor to multiple distant sites including lymph nodes, blood, liver, lung, brain, and bone.

Blood samples were collected by submandibular blood collection before placebo/immunogenic composition injection and after first, second, and third time of placebo/immunogenic composition injection. Serum from the blood samples was used for legumain IgG antibody ELISA analysis. The mouse mammary tumor cells in mice were detected by IVIS.

TABLE 1

| | | Injection | |
|---|---|---|---|
| | | volume | No. of |
| Group | Immunogenic composition | (μL) | mice |
| A | placebo | 100 | 8 |
| B | recombinant fusion protein + CpG | 100 | 10 |
| C | recombinant fusion protein + VET-SAP | 100 | 3 |

[Legumain IgG Antibody ELISA Analysis]

Experimental procedure for the legumain IgG antibody ELISA analysis: the serum of 3-10 mice (10 μL/each mouse) of group were mixed into the same tube. After diluting each group of serum 1,000-fold, 100 μL of serum dilution was added to one well of the antigen plate (coated with 1 μg/well legumain) of the Biocheck legumain ELISA kit, and reacted at 37° C. for 30 minutes. After washing 4 times with 1×PBST, 100 μL anti-mouse-IgG-HRP (1:10,000) was added and reacted at 37° C. for 30 minutes. After washing 4 times with 1×PBST, 100 μL 3,3′,5,5′-Tetramethylbenzidine (TMB) was added and reacted at room temperature for 15 minutes. After adding 100 μL of 1N H₂SO₄, the optical density at a wavelength of 450 nm was measured by an ELISA reader.

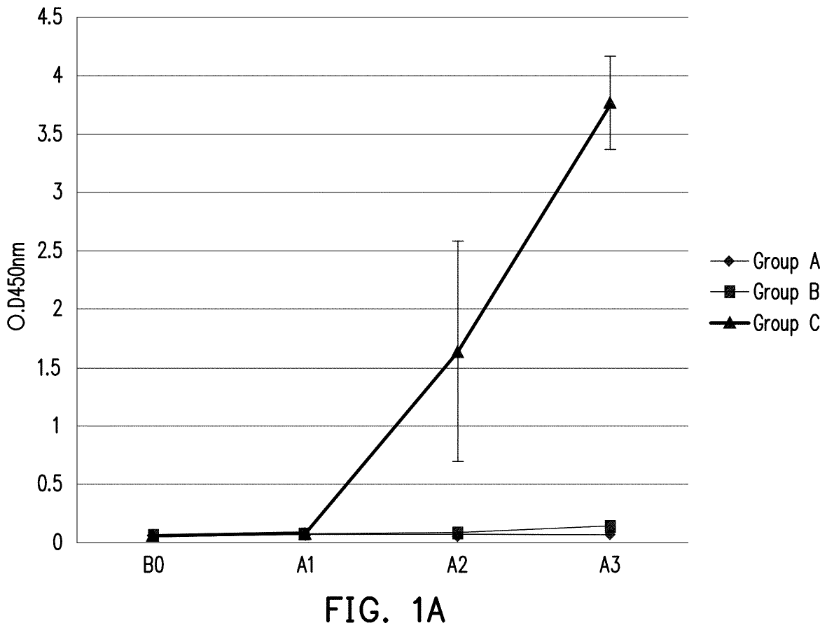

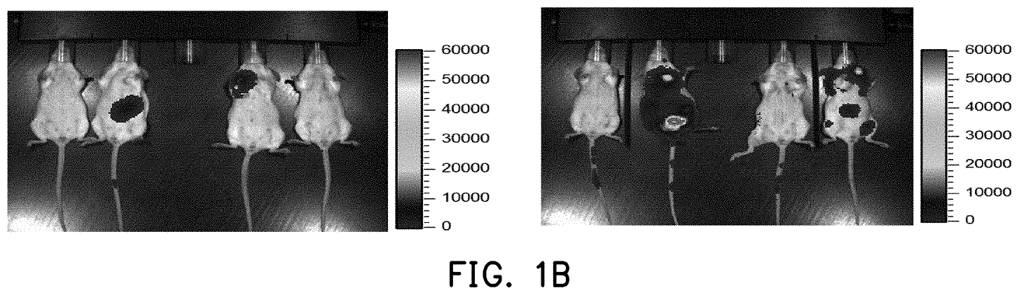

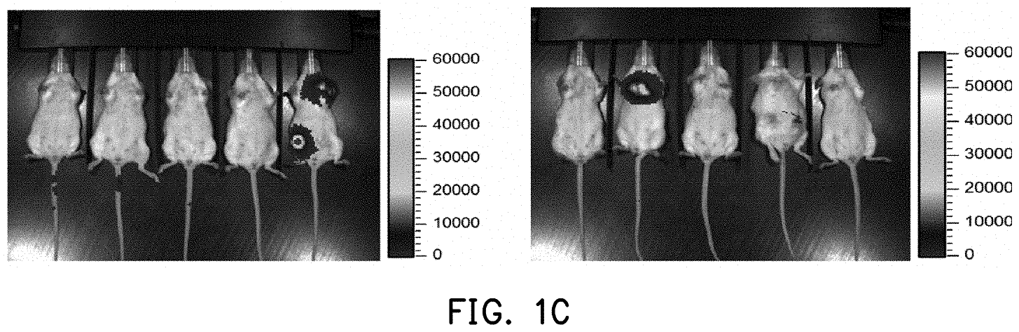

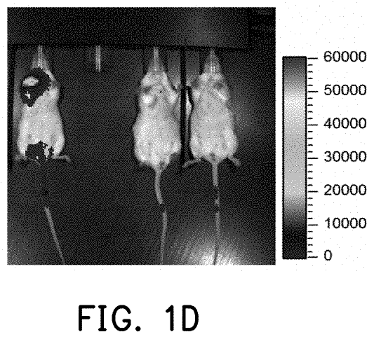

FIG. 1A is illustrating the results from ELISA for detecting the presence of legumain antibody in mice for different test groups from Example 2. The horizontal axis represents serum of each group of mice before placebo/immunogenic composition injection (B0), after first placebo/immunogenic composition injection (A1), after second placebo/immunogenic composition injection (A2), and after third placebo/immunogenic composition injection (A3). The vertical axis represents the optical density reading at a wavelength of 450 nm, which can represent the relative amount of legumain antibodies in the serum. According to the results of FIG. 1A, there was no significant change in the amount of legumain antibody from Groups A and B at A1, A2 and A3 compared to the amount of legumain antibody from Groups A and B at B0. However, at A2 and A3, the amount of legumain antibodies from Group C (injected with the immunogenic composition including the recombinant fusion protein and VET-SAP) were significantly higher than that from Groups A (injected with placebo only).

Therefore, it can be seen that the immunogenic composition including the recombinant fusion protein and VET-SAP can successfully induce specific immune responses, such as the antibody immune response, in mice with mouse mammary tumor cells.

[Detection of Tumor Cells by In Vivo Image System (IVIS)]

FIG. 1B to FIG. 1D illustrate the results from IVIS for detecting the 4T1/luc mouse mammary tumor cells in mice for different test groups from Example 2. 32 days after injection of mouse mammary tumor cells, in vivo image system (IVIS) was used to detect the signal of the 4T1/luc mouse mammary tumor cells in mice in Groups A to C. According to the results of FIG. 1B, there were 4/8 mice (about 50%) with tumor cells in the body (excluding the tail) in Group A. In FIG. 1C, there were 2/10 mice (about 20%) with tumor cells in the body (excluding the tail) in Group B. In FIG. 1D, there were ⅓ mice (about 33%) with tumor cells in the body (excluding the tail) in Group C.

Therefore, it can be seen that the immunogenic composition including the recombinant fusion protein and CpG/VET-SAP can successfully reduce the risk of cancer metastasis in mice with mouse mammary tumor cells. Moreover, in group C, immunogenic compositions comprising recombinant fusion proteins and VET-SAP can reduce the risk of cancer metastasis, at least by producing legumain antibodies. In group B, although no legumain antibodies were detected, it is believed that immunogenic compositions including recombinant fusion proteins and CpG may be triggering a cellular immune response against legumain thereby reduce the risk of cancer metastasis.

### Example 3: Effect of the Immunogenic Composition on Dog with Mammary Tumor

In this experimental example, dogs with mammary tumors were divided into 2 groups and there is 1 dog in each group as shown in Table 2. Before surgical removal of the tumor, Groups α and β were injected with the immunogenic composition including the recombinant fusion protein and VET-SAP by subcutaneous injection. Injection of the immunogenic composition was administered 3 times. After measurement of the tumor volume, the first dose of the immunogenic composition is injected in Groups α and β at Day 0, respectively. Next, the second and third dose of the immunogenic composition is respectively injected in Group α at Day 10 and 16. The second and third dose of the immunogenic composition is respectively injected in Group β at Day 10 and 16. The mammary tumor in Group α was surgically removed at Day 32.

Blood samples from Groups α and β were collected at Day 0, 10, 16, 24 and 32. Serum from the blood samples was used for legumain IgG antibody ELISA analysis. The volume of the mammary tumor in Group α was measured at Day 0, 10, 16, 24 and 32.

TABLE 2

| | | Injection | |
|---|---|---|---|
| | | volume | No. of |
| Group | Immunogenic composition | (μL) | dog |
| α | Recombinant fusion protein + VET-SAP | 1000 | 1 |
| β | Recombinant fusion protein + VET-SAP | 1000 | 1 |

[Legumain IgG Antibody ELISA Analysis]

Experimental procedure for the legumain IgG antibody ELISA analysis: after diluting each group of serum 1000-fold, 100 μL of serum dilution was added to one well of the antigen plate (coated with 0.1 μg/well legumain) of the Biocheck legumain ELISA kit, and reacted at 37° C. for 30 minutes. After washing 4 times with 1×PBST, 100 μL anti-mouse-IgG-HRP (1:10,000) was added and reacted at 37° C. for 30 minutes. After washing 4 times with 1×PBST, 100 μL 3,3′,5,5′-Tetramethylbenzidine (TMB) was added and reacted at room temperature for 15 minutes. After adding 100 μL of 1N H₂SO₄, the optical density at a wavelength of 450 nm was measured by an ELISA reader.

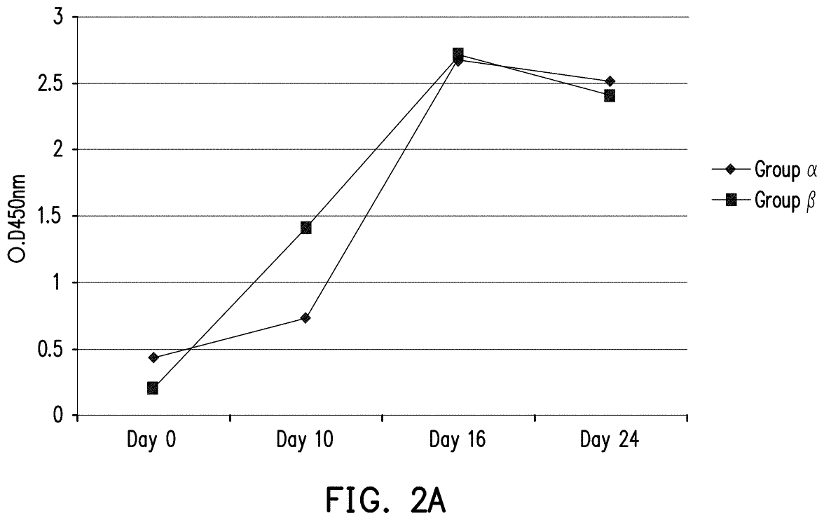

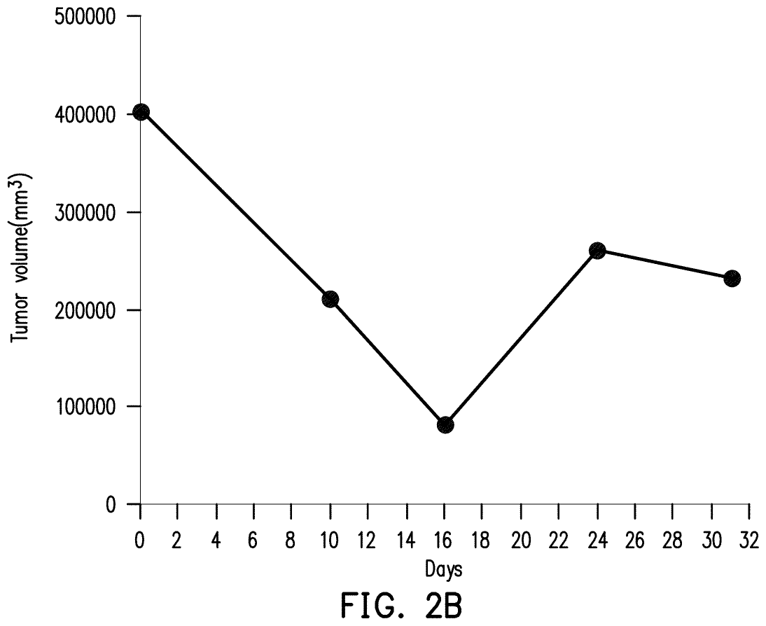

FIG. 2A is illustrating the results from ELISA for detecting the presence of legumain antibody in dogs for different test groups from Example 3. The horizontal axis represents serum of each group of dog at Day 0, 10, 16 and 24. The vertical axis represents the optical density reading at a wavelength of 450 nm, which can represent the relative amount of legumain antibodies in the serum. According to the results of FIG. 2A, the amount of legumain antibody from Groups α and β at Day 10, 16 and 24 were significantly higher than that from Groups α and β at Day 0. Therefore, it can be seen that the immunogenic composition including the recombinant fusion protein and VET-SAP can successfully induce specific immune responses, such as the antibody immune response, in dog with mammary tumor.

[Measurement of Volume of the Mammary Tumor in Group α]

FIG. 2B is illustrating the results for detecting the volume of mammary tumor in dogs from Example 3. Before injection of the first dose of the immunogenic composition, the size of the mammary tumor in Group α was measured at Day 0. After measurement, the size of the mammary tumor in Group α is about 85*95*100 mm, and the volume of mammary tumor in Group α is about 403,750 mm³ (85*95*100*½) as shown in FIG. 2B. Next, the volume of mammary tumor in Group α was also measured at Day 10, 16, 24 and 32. According to the results of FIG. 2B, after the second injection (Day 10), the tumor volume significantly became smaller. In addition, the veterinarian also observed that the tumor began to soften. Next, the tumor was surgically removed at Day 32, and after surgical removal of the tumor there is no recurrence observed. Therefore, it can be seen that the immunogenic composition including the recombinant fusion protein and VET-SAP can successfully reduce the risk of cancer metastasis in dog with mammary tumor.

According to the above embodiments, the recombinant fusion protein includes RAP1 locating at the N-terminus of the recombinant fusion protein, a CD28-PEt fusion polypeptide locating at the C-terminus of the RAP1, a legumain protein locating at the C-terminus of the CD28-PEt fusion polypeptide and a target peptide locating at the C-terminus of the legumain protein. In addition, the immunogenic composition of the present disclosure including the recombinant fusion protein is used to effectively induce specific immune responses in a subject with cancer, hence reducing the risk of cancer metastasis and recurrence for the subject.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A recombinant fusion protein, comprising:
a receptor associated protein 1 (RAP1), located at the N-terminus of the recombinant fusion protein;
a cluster of differentiation 28 (CD28)-Pseudomonas exotoxin A translocation domain (PEt) fusion polypeptide, located at the C-terminus of the RAP1;
a legumain protein, located at the C-terminus of the CD28-PEt fusion polypeptide; and
a target peptide, located at the C-terminus of the legumain protein.

2. The recombinant fusion protein according to claim 1, wherein the recombinant fusion protein comprises an amino acid sequence of SEQ ID NO: 1.

3. The recombinant fusion protein according to claim 1, wherein the recombinant fusion protein comprises an amino acid sequence encoded by a nucleotide sequence of SEQ ID NO: 2.

4. The recombinant fusion protein according to claim 1, wherein the legumain protein is a human recombinant legumain protein.

5. The recombinant fusion protein according to claim 1, wherein the target peptide is an endoplasmic reticulum retention sequence.

6. An immunogenic composition, used for inducing specific immune responses in a subject with cancer, comprising:
the recombinant fusion protein according to claim 1; and
an adjuvant.

7. The immunogenic composition according to claim 6, wherein the adjuvant comprising CpG oligodeoxynucleotides (CpG ODN) or saponin-based adjuvant.

8. The immunogenic composition according to claim 7, wherein a concentration of the CpG ODN is about 0.2 mg/ml.

9. The immunogenic composition according to claim 7, wherein a concentration of the saponin-based adjuvant is about 0.2 mg/ml.

10. The immunogenic composition according to claim 6, wherein a ratio of the recombinant fusion protein to the adjuvant is 1:1-12.5:1 by weight.

11. The immunogenic composition according to claim 6, wherein the subject comprises human and non-human animal.

12. The immunogenic composition according to claim 6, wherein the cancer comprises legumain-overexpressed cancer.

13. The immunogenic composition according to claim 12, wherein the legumain-overexpressed cancer comprises breast cancer, mammary tumor, prostate cancer, stomach cancer, colorectal cancer, ovarian cancer or melanoma.


SEQUENCE LISTING (2 sequences)

SEQ ID NO: 1 - protein, 492 aa, Homo sapiens
MAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRAREFTDIYFCKIEVMYPPPYLDNEKSNGTIIHRARYKRGWEQLEQCGYPVQRLVALYLAARLSWNQVDQVIRGSEFDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNLEKDELKDELKDEL

SEQ ID NO: 2 - DNA, 1473 bp, Homo sapiens
gctgagttcgaggagccgcgtgtgattgacctgtgggacctggcgcagtccgccaacctcacggacaaggagctggaggcgttccgtgaggagctcaagcacttcgaagccaaaatcgagaagcacaaccactaccagaagcagctggagattgcgcacgagaagctgcgtcacgcagagagcgtgggcgacggcgagcgtgtgagccgcagccgcgagaagcacgccctgctggagggccgtaccaaggagctgggctacacggtgaagaagcatctgcaggacctgtccggccgtatctcccgtgctcgtgaattcaccgacatctacttctgcaaaatcgaagttatgtacccgccgccgtacctggacaacgaaaaatctaacggtaccatcatccaccgtgcgcgttacaaacgtggttgggaacagctggaacagtgcggttacccggttcagcgtctggttgcgctgtacctggcggcgcgtctgtcttggaaccaggttgaccaggttatccgtggttctgaattcgacggtggtaaacactgggttgttatcgttgcgggttctaacggttggtacaactaccgtcaccaggcggacgcgtgccacgcgtaccagatcatccaccgtaacggtatcccggacgaacagatcgttgttatgatgtacgacgacatcgcgtactctgaagacaacccgaccccgggtatcgttatcaaccgtccgaacggtaccgacgtttaccagggtgttccgaaagactacaccggtgaagacgttaccccgcagaacttcctggcggttctgcgtggtgacgcggaagcggttaaaggtatcggttctggtaaagttctgaaatctggtccgcaggaccacgttttcatctacttcaccgaccacggttctaccggtatcctggttttcccgaacgaagacctgcacgttaaagacctgaacgaaaccatccactacatgtacaaacacaaaatgtaccgtaaaatggttttctacatcgaagcgtgcgaatctggttctatgatgaaccacctgccggacaacatcaacgtttacgcgaccaccgcggcgaacccgcgtgaatcttcttacgcgtgctactacgacgaaaaacgttctacctacctgggtgactggtactctgttaactggatggaagactctgacgttgaagacctgaccaaagaaaccctgcacaaacagtaccacctggttaaatctcacaccaacacctctcacgttatgcagtacggtaacaaaaccatctctaccatgaaagttatgcagttccagggtatgaaacgtaaagcgtcttctccggttccgctgccgccggttacccacctggacctgaccccgtctccggacgttccgctgaccatcatgaaacgtaaactgatgaacaccaacctcgagaaagacgaactgaaggatgagctcaaagacgaactg